(19) United States
(12) Patent Application Publication
(10) Pub. No.: US 2005/0115573 A1
(43) Pub. Date: Jun. 2, 2005

(54) Physically aggressive criminal control and restraint system, "The Giraffe"

(76) Inventor: Stephen Dintino, Coatesville, PA (US)

Correspondence Address: STEPHEN DINTINO, 913 GREENWOOD CIRCLE, COATESVILLE, PA, 19320, US

(21) Appl. No.: 10/725,907

(22) Filed: Dec. 2, 2003

(51) Int. Cl. A61B 19/00

(52) U.S. Cl. 128/869; 128/876

(57) ABSTRACT

The Physically Aggressive Criminal Control and Restraint System, “The Giraffe”, is a new and unique manual protective control and restraint tool, which will safely control and restrain a physically aggressive person, and which will provide the necessary distance from the physically aggressive person to protect the users of the tool. The tool is specifically and intentionally designed to be used by Public Safety personnel.

The Physically Aggressive Criminal Control and Restraint System, “The Giraffe”, is a tool and system to be used by Police, Law Enforcement, Corrections, Security, Medical and Public Safety personnel to safely control and restrain a physically aggressive criminal or emotionally or psychologically unstable person, threatening to injure or kill himself, herself or other people.

“The Giraffe” is intended to be used by one or two Public Safety personnel who will hold and direct the tool around the arms and legs of the physically aggressive person, which will safely influence the control and restraint of the physically aggressive person, and which will provide the distance necessary to protect the Public Safety personnel from the physically aggressive person. “The Giraffe” is currently being used by the Tredyffrin Township Police Special Operations Team, Chester County, Pennsylvania.

“The Giraffe” is a reinforced one and a half (1.5) inch PVC plastic or one (1) inch tubular aluminum collapsible extension, folding, or attachable and detachable pole, with three (3) sets of handles and one (1) shoulder support at the users' end, and two (2) sets of control and restraint arms at the recipient's end, ninety-six (96) inches in length overall, sixteen (16) inches in height at the users' end, thirty-six (36) inches in height at the control and restraint arms recipient's end, and thirty-four (34) inches in width at the control and restraint arms recipient's end. Elastic rubber bands (rubber bungee straps) connect the control and restraint arms.

### DESCRIPTION OF DRAWING

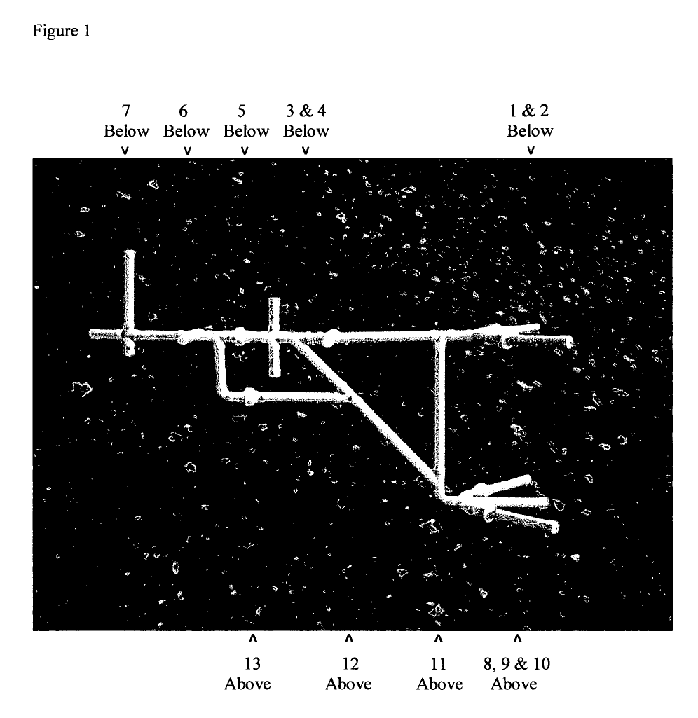

An illustration of “The Giraffe” is separately attached, which is identified as “FIG. 1”, and which is listed with the descriptive parts. The illustration scale is: forty-four thousandths (0.044) inch equals one (1) inch.

The illustration of “The Giraffe” is numbered and identified.

The overall length of “The Giraffe” is 96 inches.

1 & 2: Two upper control and restraint arms, with detachable, folding or expandable joints. The two arms are 25 inches long, and the two arms are 34 inches apart at the ends. A rubber elastic band (rubber bungee strap) is hooked and stretched between the ends of the arms. The upper control and restraint arms are 36 inches apart from the lower control and restraint arms (8, 9 & 10), which are connected by the vertical connector (11).

3 & 4: One person user horizontal and vertical handles. The handles are 7 inches long on each side of the pole. The one person user horizontal handle is 50 inches from the end of the upper control and restraint arms (1 & 2). The one person user vertical handle (3) is 12 inches from the one person user horizontal handle (4).

5: Pole detachable, folding or expandable joint. The pole joint is 6 inches from the one person user vertical handle (4).

6: Two person user horizontal handle. The handles are 7 inches long on each side of the pole. The two person user horizontal handle is 12 inches from the one person user vertical handle (4).

7: Two person user vertical shoulder support and handle. User's end. The two person user vertical shoulder support and handle is 16 inches long, and is 12 inches from the two person user horizontal handle.

8, 9 & 10: Three lower control and restraint arms, with detachable, folding or expandable joints. The two outer arms are 25 inches long, and the two outer arms are 34 inches apart at the ends. The center arm is 22 inches long, and is 16.25 inches from each outer arm. A rubber elastic band (bungee strap) is hooked and stretched between the ends the arms.

11: Vertical connector for the upper and lower control and restraint arms. The vertical connector is 34 inches long, and connects the upper and lower control and restraint arms.

12: Diagonal support for the vertical connector (11) and the pole. The diagonal support is 42 inches long, and connects the vertical support and the pole.

13: Diagonal support detachable, folding or expandable joint.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISK APPENDIX

Not applicable

### BACKGROUND OF THE INVENTION

The Physically Aggressive Criminal Control and Restraint System, “The Giraffe”, is a tool and system to be used by Police, Law Enforcement, Corrections, Security, Medical and Public Safety personnel to safely control and restrain a physically aggressive criminal or emotionally or psychologically unstable person, threatening to injure or kill himself, herself or other people.

“The Giraffe” is intended to be used by one or two Public Safety personnel who will hold and direct the tool around the arms and legs of the physically aggressive person, which will safely influence the control and restraint of the physically aggressive person, and which will provide the distance necessary to protect the Public Safety personnel from the physically aggressive person.

“The Giraffe” is currently being used by the Tredyffrin Township Police Special Operations Team, Chester County, Pennsylvania.

### BRIEF SUMMARY OF THE INVENTION

In certain specific situations and incidents, “The Giraffe” can be used to safely control and restrain a physically aggressive person by controlling the subject's arms and legs with the Giraffe's rubber banded arms. The physically aggressive person can be restrained against a wall, or in the absence of a wall, the subject can be restrained by turning and rotating “The Giraffe” to place the subject on the floor or ground.

“The Giraffe’ is intended to be used by trained personnel and in conjunction and in coordination with the use of distractions and less lethal force, and an element of subject control and restraint handlers. A comprehensive “Less Lethal Tactical Intervention Tactic” training and instruction course is provided and coordinated with the use of “The Giraffe”.

### SEQUENCE LISTING

Not applicable

### NON-PROVISIONAL UTILITY PATENT APPLICATION SMALL ENTITY FEE

I, Stephen Dintino, sole inventor and applicant, declare and affirm that I am a “small entity”, and I respectfully petition the United States Patent and Trademark Office to approve the patent of my invention, the Physically Aggressive Criminal Control and Restraint System, “The Giraffe”. I submit the applicable fee of $385 for the processing of this patent application.

1. I, Stephen Dintino, claim that I solely invented, initiated, developed and made the Physically Aggressive Criminal Control and Restraint System—“The Giraffe” for the intent purpose and need to provide Police, Law Enforcement, Corrections, Medical and Public Safety personnel with an unique tool to safely control and restrain a physically aggressive person, and to minimize the use of force.
